(19) United States
(12) Plant Patent Application Publication
(10) Pub. No.: US 2004/0154063 P1
(43) Pub. Date: Aug. 5, 2004

(54) HYDRANGEA HYBRID PLANT NAMED 'SWEET CHRIS'

(76) Inventor: Joseph Gray, Winters, CA (US)

Correspondence Address: CHRISTIE, PARKER & HALE, LLP, P.O. BOX 7068, PASADENA, CA, 91109-7068, US

(21) Appl. No.: 10/357,938

(22) Filed: Feb. 3, 2003

(51) Int. Cl. A01H 5/00

(52) U.S. Cl. PLT/250

(57) ABSTRACT

A new plant variety of Hydrangea hybrid characterized by dark green foliage, burgundy fall foliage, lacecap flower color and burgundy stem color.

### BACKGROUND OF THE INVENTION

The present invention relates to a new and distinct variety of Hydrangea hybrid which was developed in a controlled breeding program in Silver Spring, Md., United States, by Mr. Joseph Gray. The varietal denomination of the new variety is ‘Sweet Chris’.

The genus Hydrangea is included in the family Saxifragaceae which comprises about 80 genera and 1,200 species of herbs, shrubs, small trees and woody climbers mostly temperate in origin. Hydrangea comprises approximately 23 species of deciduous or evergreen shrubs and vines originating in North America, South America and East Asia, many of which possess desirable ornamental characteristics. Recent taxonomical treatments sometimes isolate Hydrangea in a new family, Hydrangeaceae.

Hydrangea macrophylla is widespread and common throughout much of Japan. It generally has opposite, simple leaves, obovate to elliptic in shape, 10 to 20 cm long and 5 to 15 cm wide. Leaf color ranges from light to dark green, depending on light intensity and cultural conditions. Leaves are generally softly pubescent. Flower color is dependent upon the pH level of the soil and the amount of available aluminum in the soil media. Flowers of Hydrangea macrophylla are borne in mostly sterile florets, formed in what is often called a “hortensia” flower.

Hydrangea serrata is widespread and common throughout much of Japan, Korea and China. It generally has opposite, simple leaves, lanceolate in shape with acuminate apices, 5 to 15 cm long and 5 to 10 cm wide. Leaf color ranges from light to dark green, depending on light intensity and cultural conditions. Leaves are generally softly pubescent. The plant is deciduous. Flower color is dependent upon the pH level of the soil and the amount of available aluminum in the soil media. Flowers of Hydrangea serrata are borne in mostly flattened corymbs, 1 to 5 cm wide, with up to 12 sterile florets.

### SUMMARY OF THE INVENTION

The new variety was discovered in a controlled breeding program of Hydrangea macrophylla and Hydrangea serrata, and differs from its parents in its dark green foliage, burgundy fall foliage, lace cap flower color, and burgundy stem color. Asexual reproduction of the new variety by stem cuttings, performed in Silver Springs, Md. and Vacaville, Calif., have confirmed that the distinctive characteristics of the new variety are stable and transmitted to succeeding generations, and the new variety reproduces true to type.

### COMPARISON WITH PARENTS

‘Sweet Chris’ is distinguished from its parents and all other varieties of Hydrangea of which I am aware by dark green foliage, burgundy fall foliage, lacecap flower color, and burgundy stem color.

### BRIEF DESCRIPTION OF ILLUSTRATION

The accompanying illustrations show a specimen of the new cultivar in a photo illustration as true to color as is reasonably possible to make in an illustration of this character.

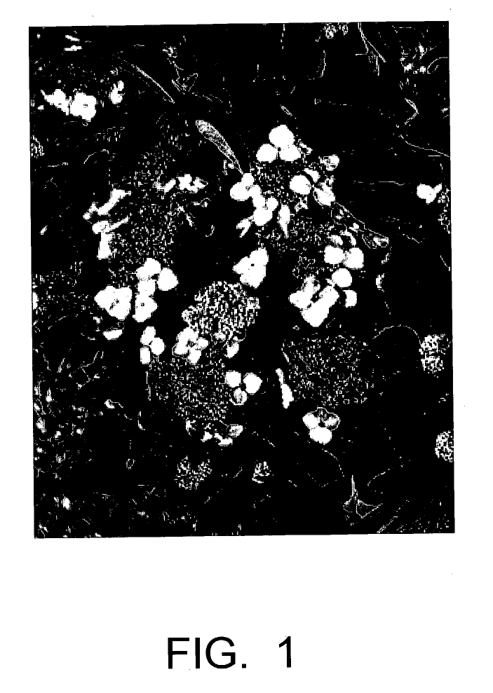

FIG. 1 illustrates the inflorescence and overall habit of ‘Sweet Chris’

### DETAILED DESCRIPTION OF THE NEW VARIETY

‘Sweet Chris’ has not been observed under all possible environmental, cultural and light conditions. The following observations and descriptions are of plants grown in Vacaville, Calif. In this description, color references are to The Royal Horticultural Society Colour Chart (1995) and terminology used in the color descriptions herein refers to plate numbers in this color chart. Phenotypic expression may vary with light intensity, cultural and enviromnental conditions.

### CLASSIFICATION

Botanical: Hydrangea (Thunb.) Ser. ‘Sweet Chris’.

Parentage:

Mother plant.—Hydrangea macrophylla ‘Lanarth White’.

Father plant.—Hydrangea serrata ‘Blue Billow’.

Propagation.—By vegetative cuttings.

The following plant descriptors and color readings are of mature specimens grown in Vacaville, Calif. under 50% shade, in a 6.6 pH soiless nursery growing media.

### PLANT

Size: 2.5 meters.

Habit: Upright, compact shrub.

Branching:

Lateral branches.—Approximately 30.

Typical # branches.—30.

Observed # branches.—20.

Size.—2.5 meters.

Diameter: 7 mm.

Lenticles.—Near Red-Purple Group 59C.

Branch color.—June-September: Near Red Purple Group 59C April-May; October-November: Near Green Group 138C.

### LEAF

Shape: Ovate to broadly ovate, with a cuneate base and acute to acuminate apex.

Leaf size: To 130 mm long and 80 mm wide.

Arrangement: Opposite.

Margin: Serrate.

Texture/Substance: Firm to coriaceous glabrous to nearly dull above, glabrous beneath, veins depressed above and raised beneath.

Color:

Young foliage.—Top and Bottom near Yellow-Green Group 149A.

Mature foliage.—Upper side: Near Yellow-Green Group 146B Under side: Near Yellow-Green Group 144B.

Venation:

Pattern: Alternately pinnate, sunken above, raised beneath.

Coloration.—New Foliage: Upper Surface: Near Green Group 138B Under Surface: Near Green Group 138B Mature Foliage: Upper Surface: Near Green Group 139A Under Surface: Near Green Group 139A.

Fall foliage: Upper Surface: Near Greyed-Purple Group 187A.

Petioles:

Size.—2.5 to 3 cms long.

Coloration.—Upper Surface: Near Red Group 59C Under Surface: Near Green Group 138D.

Texture.—Smooth.

Internodes: 25 mm.

Hardiness: USDA Zone 6 (−10° F. to 0° F.).

Vigor: Vigorous growth.

Pests/Diseases: None noted.

Soil pH: 6.6 pH in a soiless nursery growing media.

### INFLORESENCE

Bloom period:

Outdoors.—April to September

Greenhouse.—Not observed in a greenhouse.

Flower form: Paniculate, compound corymb inflorescence; both sterile (65), sepalous florets and fertile (10), asepalous florets borne on same panicle.

Flower arrangement: Loose tiered corymb 15 to 18 cm across.

Size of inflorescence: 18 cms.

Shape of inflorescence: Hemispherical.

Fragrance: None.

Lastingness of individual blooms: Flowers are very persistent, lasting up to 5 months. As the flowers mature, the pedicels bearing the outer florets bend to invert the florets toward the ground.

Sepals:

Number.—Usually 4 sepals per floret, occasionally 3.

Shape.—Broadly ovate.

Size.—Variable; typical sepal measures 20 mm by 25 mm.

Margin.—Dentate to crenate on the upper half of sepal.

Curvature.—Reflexed.

Arrangement.—Overlapping at base to midpoint.

Color.—Bicolored; centers near Red-Purple Group 58B in early bloom, maturing to near Red-Purple Group 63A and eventually 61B; margins near Violet Group 83B.

Size of one large 4-petaled floret.—About 3 cm wide.

Aspect of sepals.—Smooth.

Non-sepalous florets:

Size of floret.—Approximately 2 mm in diameter.

Color of floret.—Near Red-Purple Group 63B.

### REPRODUCTIVE ORGANS

Stamens: Approximately 8.

Stigma: Usually 4, occasionally 3 or 5.

Pollen amount: Very little, not measurable.

Pollen color: Near White Group 155D.

Fertile inflorescence:

Pistil length.—2 mm.

Stigma color.—Near Red-Purple Group 57D.

Style color.—Near Red-Purple Group 57D.

Ovary color.—Near Red-Purple Group 57D.

Stamen filament.—Near Red-Purple Group 57C.

Fruit.—None noted.

I claim:

1. A new plant variety of Hydrangea hybrid of the variety substantially as shown and described.
